(19) United States
(12) Patent Application Publication
(10) Pub. No.: US 2005/0103097 A1
(43) Pub. Date: May 19, 2005

(54) Sensor

(76) Inventors: Carsten Faltum, Fredensborg (DK); Jacob Thaysen, Copenhagen (DK)

Correspondence Address: JACOBSON HOLMAN PLLC, 400 SEVENTH STREET N.W., SUITE 600, WASHINGTON, DC, 20004, US

(21) Appl. No.: 10/501,911

(22) PCT Filed: Jan. 24, 2003
(86) PCT No.: PCT/DK03/00042

Related U.S. Application Data
(60) Provisional application No. 60/354,524, filed on Feb. 8, 2002.

(30) Foreign Application Priority Data
Jan. 24, 2002 (DK) PA 2002 00125

(51) Int. Cl. G01N 33/00; G01N 31/00

(52) U.S. Cl. 73/61.41; 73/64.53; 422/68.1

(57) ABSTRACT

The invention relates to a sensor for detecting a substance in a liquid. The sensor comprises a pillar shaped primary substrate and a sensor unit, e.g. a cantilever connected to the primary substrate. The sensor comprises detecting means e.g. in the form of a piezoresistive element, a strain gauge, a Si or C nanotube, a capacitor or a piezoresistor, for detecting a change of stress or mass generated on a surface area of the sensor unit, and an electric communication line for applying a voltage over said detection means, wherein at least one of the wires is integrated in the pillar shaped primary substrate. The sensor in the form of a cantilever may e.g. have a two-dimensional shape selected from the group consisting of square, rectangular, triangular, pentagonal, hexagonal, leaf shaped, circular and oval periphery. The primary substrate may be connected to a secondary substrate such as an electronic chip comprising contact pads corresponding with wire exits from the primary substrate.

### FIELD OF THE INVENTION

The present invention relates to a sensor comprising one or more sensor units, wherein each sensor units comprises detection means for detection of stress or mass generated on a surface area of the sensor unit or units, e.g. in the form of a surface stress sensing element or a mass sensing element and wires connected to the detection means. The type of sensor unit most commonly used is a cantilever.

### BACKGROUND OF THE INVENTION

In the prior art, such cantilevers have been used in atomic force microscopy technology (AFM) and in the art of detecting components in fluids such as gas and liquids.

It is known from e.g. WO 0066266 and WO 9938007 that micro cantilevers can be used for detection of molecular interaction. Capture molecules are immobilised on the surface of the cantilever. The capture molecule can basically be any molecule that specifically binds to another molecule. Capture molecules can be DNA oligoes, proteins, antigen, antibodies, ligands, etc. When the capture molecules bind to an analyte in the sample that is presented to the cantilever, this will induce a change in the surface stress of the cantilever, and consequently the cantilever will deflect or stretch.

By measuring the reflection angle of a laser beam that is directed to the cantilever, the deflection can be detected. This principle is also known from the atomic force microscopy (AFM). Another detection principle is the use of a piezoresistor integrated into the cantilever for detecting the surface stress directly. In this detection principle the deflection is detected as a change in the electrical resistance of the piezoresistor.

From stress formation studies in ambient and aqueous environments, micrometer-sized cantilevers with optical read-out have proven very sensitive as described in the articles Berger, R., Gerber, Ch., Lang, H. P. & Gimzewski, J. K. Micromechanics: A toolbox for femtoscale science: “Towards a laboratory on a tip”. Microelectronic Engineering. 35, 373-379 (1997), and O'Shea, S. J., Welland, M. E. Atomic force Microscopy stress sensors for studies in liquids. J. Vac. Sci. Technol. B. 14, 1383-1385 (1996).

Basically, a biochemical reaction at the cantilever surface can be monitored as a bending of the cantilever due to a change in the surface stress. Surface stress changes in self-assembled alkanethiols on gold have earlier been measured in air by this technique, and surface stress changes of approximately 10⁻⁵ N/m can be resolved by cantilever-based methods. This sensor principle has a wide range of applications in the detection of specific biomolecules as well as in real time local monitoring of chemical and biological interactions.

Cantilever-based sensors with integrated piezoresistive read-out are described by Thaysen, J., Boisen, A., Hansen, O. & Bouwstra, S. AFM probe with piezoresistive read-out and highly symmetrical Whetstone bridge arrangement. Proceedings of Transducers′99, 1852-1855 (Sendai 1999). Hereby the stress changes on the cantilever sensors can be registered directly by the piezoresistor. Moreover, integrated read-out greatly facilitates operation in solutions since the refractive indices of the liquids do not influence the detection. Each sensor has a built-in reference cantilever, which makes it possible to subtract background drift directly in the measurement. The two cantilevers are connected in a Whetstone bridge, and the stress change on the measurement cantilever is detected as the output voltage from the Whetstone bridge.

In the prior art realisations of cantilever-based sensors with piezoresistive read-out, the piezoresistors are contacted through ‘horizontal’ wiring on the sensor chip, i.e. the electrical wires are placed parallel to the surface of the cantilever arm and the surface of the substrate carrying the cantilever. FIG. 1 shows such prior art cantilever. The wiring takes up a considerable amount of space and is therefore a crucial limiting factor when trying to maximise the number of cantilevers on a sensor chip. Furthermore, the horizontal wiring makes it very difficult to build two dimensional arrays of cantilever sensors. As described in WO 0066266, it is feasible to place two rows of cantilever sensors facing each other, but if additional rows of cantilevers are added the spacing between adjacent rows will increase significantly due to the electrical wiring. Moreover, it is not possible to place more than two rows of cantilevers in a liquid handling system. That is, cantilevers on either side of a liquid channel.

### SUMMARY OF THE INVENTION

The objective of the present invention is to provide a sensor suitable for use in the detection of one or more components in a liquid, which sensor has an improved design.

In particular, one objective of the invention to provide a sensor which can be incorporated into or constitute a microchip and wherein two or more sensor units comprising wires for applying a voltage can be incorporated, thereby overcoming the wiring problem discussed above.

A further objective of the present invention is to provide a sensor of micrometer dimensions comprising one or more sensor units, which allows freedom of design with respect to the sensor and freedom of positioning with respect to the sensor units.

These and other objectives have been achieved by the invention as defined in the claims.

### DISCLOSURE OF THE INVENTION

The sensor according to the invention comprises a pillar shaped primary substrate and at least one sensor unit connected to the pillar shaped primary substrate.

Normally the pillar shaped primary substrate and the sensor unit will be made in one piece, e.g. using photo resist technology as it is generally known. But the sensor unit and the pillar shaped primary substrate may be prepared separately and thereafter connected to each other, e.g. using welding techniques, glue or other well-known techniques.

In the following the sensor is described with one sensor unit, but it should be understood that the sensor usually has more than one sensor unit. Similarly the sensor is described with one primary pillar shaped substrate, but the sensor unit may naturally comprise several or even a plurality such as up to 10, up to 50 or even up to 100 primary pillar shaped substrates.

The sensor unit comprises means for detecting a change of stress and/or mass generated on a surface area of the sensor unit(s), and an electric communication line for applying a voltage over said detection means.

In one embodiment, the surface stress, in the form of a deflection or in the form of a change in resonance frequency in case the sensor unit is amplified, may be measured using laser technology as known from the prior art technology, e.g. as described in WO 0066266, WO 0058729, U.S. Pat. No. 6,016,686 and U.S. Pat. No. 6,289,717, the teachings of laser detection of cantilever deflections and/or amplification frequencies hereby being incorporated by reference as useful in one or more of the embodiments of the present invention.

In one embodiment, the detection means is in the form of a laser system comprising a laser capable of directing a laser beam towards the sensor unit, and means for detection of the reflection angle of the laser beam.

In one embodiment, the detection means is in the form of a surface stress sensing element. The surface stress sensing element may preferably be incorporated in the sensor unit so that liquid does not come into direct contact with the surface stress sensing element when the sensor unit is in contact with the liquid. Such surface stress sensing element is generally known in the art and includes in particular a surface stress sensing element that acts by applying a voltage over the stress sensing element so that the surface stress can be measured as a change in resistance, a change in capacity or other changes of electric signal.

The surface stress sensing element may e.g. be a capacitor and/or a piezoresistor and/or a piezoelectric element and/or a strain gauge. The capacitor may be in the form of two conducting elements of e.g. metal or conductive polymers separated a short distance e.g. between 0.5 and 3 μm from each other by a dielectricum such as liquid dielectricum, gas dielectricum or solid dielectricum e.g. air, or octafunctional epoxidized novalac e.g. SU-8.

In one embodiment, the detection means is in the form of a piezoelectric element which may e.g. be a surface stress sensing element, but may also be a mass sensing element. The mass stress sensing element may preferably be incorporated in the sensor unit so that liquid does not come into direct contact with the mass sensing element when the sensor unit is in contact med the liquid.

The piezoelectric element may e.g. be of a material selected from the group consisting of quartz, PZT, PVDF, ZnO or solgel. In one embodiment, the piezoelectric unit is in the form of a film. The piezoelectric film can be suitably made of piezoelectric ceramics, but may be made of electrostrictive ceramics or ferro-electric ceramics. The piezoelectric film may be made of a material required to be subjected to a polarization process or a material not required to be subjected to the polarization process.

The ceramics used for the piezoelectric film may e.g. be ceramics containing lead zirconate, lead magnesium niobic acid, lead nickel niobic acid, lead zinc niobic acid, lead manganin niobic acid, lead antimonic stannic acid, lead titanium acid, lead manganic tungsten acid, lead cobalt niobic acid, barium titanate or the like, or containing the components of any combination of the above material.

Also, the piezoelectric film may be of the above ceramics to which oxide of lanthanum, calcium, strontium, molybdenum, tungsten, barium, niobium, zinc, nickel manganin or the like, any combination of the above material, or other compounds are appropriately added. For example the film may be of ceramics which mainly contain components consisting of lead magnesium niobic acid, lead zirconate, lead titanium acid in addition to ceramics containing lanthanum or strontium. The piezoelectric film may e.g. be prepared as described in U.S. Pat. No. 5,892,143. The thickness of the piezoelectric film is e.g. 1 to 100 μm, such as 5 to 50 μm. When the thickness of the piezoelectric film exceeds 100 μm, the sensitivity may be deteriorated, and when it is less than 1 μm, it may be difficult to ensure the reliability. When a voltage is applied over the piezoelectric element to vibrate the piezoelectric element, the resonance frequency will depend on the mass to be vibrated. Thereby it can be detected if a target component has been captured by the surface of the sensor unit into which the piezoelectric element is integrated.

In one embodiment, where the stress sensing element is a piezoresistor or a strain gauge, the piezoresistor may comprise or preferably consist of a material selected from the group consisting of amorph silicon, polysilicon, single crystal silicon, metal or metal containing composition, e.g. gold, AlN, Ag, Cu, Pt and Al conducting polymers, such as doped octafunctional epoxidized novalac e.g. doped SU-8, and composite materials with an electrically non-conducting matrix and a conducting filler, wherein the filler preferably is selected from the group consisting of amorph silicon, polysilicon, single crystal silicon, metal or metal containing composition e.g. gold, AlN, Ag, Cu, Pt and Al, semi-conductors, carbon black, carbon fibres, particulate carbon, carbon nanowires, silicon nanowires. As used herein, a “nanotube” is a nanowire that has a hollowed-out core and includes those nanotubes know to those of ordinary skill in the art. A “non-nanotube nanowire” is any nanowire that is not a nanotube. Further information about useful nanowires can be found in WO 0248701 which is hereby incorporated by reference.

In one embodiment, it is preferred that the surface stress sensing element is a piezoresistor. Such piezoresistor is well known in the art and is e.g. described in the following publications which are hereby incorporated by reference: U.S. Pat. No. 6,237,399, U.S. Pat. No. 5,907,095, Berger, R. et al. Surface stress in the self-assembly of alkanethiols on gold. Science. 276, 2021-2024 (1997); Berger, R., Gerber, Ch., Lang, H. P. & Gimzewski, J. K. Micromechanics: A toolbox for femtoscale science: “Towards a laboratory on a tip”. Microelectronic Engineering. 35, 373-379 (1997); Thaysen, J., Boisen, A., Hansen, O. & Bouwstra, S. AFM probe with piezoresistive read-out and highly symmetrical Whetstone bridge arrangement. Proceedings of Transducers′99, 1852-1855 (Sendai 1999); Boisen A., Thaysen J., Jensenius H., & Hansen, O. Environmental sensors based on micromachined cantilevers with integrated read-out. Ultramicroscopy, 82, 11-16 (2000).

The pillar shaped primary substrate may in principle be of any type of material, such as one or more of the materials selected from the group consisting of silicon, silicon nitride, silicon oxide, metal, metal oxide, glass and polymer, wherein the group of polymers preferably includes epoxy resin, polystyrene, polyethylene, polyvinylacetate, polyvinylcloride, polyvinylpyrrolidone, polyacrylonitrile, polymethylmetacrylate, polytetrafluoroethylene, polycarbonate, poly-4-methylpentylene, polyester, polypropylene, cellulose, nitrocellulose, starch, polysaccharides, natural rubber, butyl rubber, styrene butadiene rubber and silicon rubber.

In order to have optimal processability, the primary substrate should preferably be of or comprise a material which can act as a photo resistor. Preferred materials include an epoxy resin, preferably selected from the group consisting of epoxy functional resin having at least two epoxy groups, preferably an octafunctional epoxidized novalac. Particularly preferred materials are described in U.S. Pat. No. 4,882,245, which is hereby incorporated by reference. The most preferred material is the octafunctional epoxidized novalac which is commercially available from Celanese Resins, Shell Chemical, MicroChem Inc under the tradename SU-8, and from Softec Microsystems under the tradename SM10#0.

Basically, it is preferred that the sensor unit is based on a material included in the pillar shaped primary substrate or preferably on the same material as that of the pillar shaped primary substrate. If the sensor unit and the pillar shaped primary substrate are made in one piece, it is naturally based on the same material, but the sensor unit and the pillar shaped primary substrate may include one or more layers of material not included in the other part.

In one embodiment, the sensor unit and the pillar shaped primary substrate are based on the same material so that the major parts of the material of these respective parts are of the same material.

If the sensor unit and the pillar shaped primary substrate are made in separate pieces and connected afterwards, the material should at least be compatible with each other, and preferably the major weight part of the materials should be identical.

For applications in liquid, the wires need to be insulated, and the pillar shaped primary substrate should therefore preferably consist of or comprise an electrically insulating material, which prevents short-circuiting of the electrical connections during operation. The insulating material could e.g. be a polymer, silicon nitride, silicon oxide, metal oxides, etc. In case the electrical connection line includes doped silicon, the insulating property can be obtained by reversed biased diode effect. For a wire consisting of p-type silicon, the reversed biased diode effect is obtained by encapsulating the wire in n-type silicon.

The sensor according to the invention includes an electric communication line for applying a voltage over the detection means.

In one embodiment, the detection means is a surface stress sensing element.

In one embodiment, the sensor according to the invention includes an electric communication line for applying a voltage over the surface stress sensing element or the mass sensing element. In the following the configuration, materials and other information concerning the communication line are described with respect to the surface stress sensing element, but it should be understood that the description also includes a description of a communication line over a mass sensing element of embodiments containing a mass sensing element.

The electric communication line includes a pair of wires connected to the surface stress sensing element. The electric wire may be of the same material as the surface stress sensing element, particular if the surface stress sensing element is a piezoresistor. In the situation where the surface stress sensing element and the piezoresistor are of the same material, the piezoresistor will preferably be thinner, e.g. a thinner layer or a smaller wire diameter. In other situations the surface stress sensing element and the piezoresistor are of different materials and are fixed to each other at a connection point e.g. by welding. The method of connecting wires to a surface stress sensing element is generally known in the art, and reference is made to the prior art referred to above. The electric communication line may consist of the wires, but it may also include other elements such as diodes, other resistors, e.g. a part of a Whetstone bridge or other surface stress sensing elements of the sensor.

At least one of the wires of the pair of wires is integrated in the pillar shaped primary substrate. If several wires are integrated in the pillar shaped primary substrate, the wires should be separated by insulating material to prevent short circuiting.

A high degree of design freedom may e.g. be achieved by integrating the wire(s) in the pillar shaped primary substrate, since the primary substrate is shaped as a pillar and at least one of said wires is integrated in said pillar shaped primary substrate. The pillar shaped primary substrate is e.g. protruding from a carrier substrate (a secondary substrate) so as to form a channel extending around the pillar shaped substrate.

In the following any positions of the sensor unit or the surface stress sensing element/mass sensing refer to the sensor unit/surface stress sensing element in a non-stressed state.

A further high degree of design freedom may e.g. be achieved by integrating at least two wires in the primary substrate so that at least two wires pass through the material of the primary substrate.

It is preferred that other optional elements of the electric communication line are not integrated in the pillar shaped primary substrate, but in some embodiments it may be convenient to at least partly integrate one or more of other elements of the electric communication line into the primary substrate. This may e.g. reduce the size of the sensor.

The term “integrated in the primary substrate” as used herein means that the wire is embedded in the primary substrate material for at least a length of the wire.

The wire may e.g. be integrated in the primary substrate by passing through a channel in the pillar shaped primary substrate.

The sensor unit may in principle be any type of flexible unit which is usable in connection with surface stress or mass sensing elements. Generally, it is preferred that the sensor unit is a flexible sheet-formed unit having an average thickness thinner than both its average thickness and its average width. Such sensor units preferably include cantilevers, bridges and diaphragms. In principle, however, the sensor unit may also be shaped as a cord.

The thickness of the sensor unit may preferably be between 0.1 and 25 μm, more preferably between 0.3 and 5 μm, such as about 1 μm. The other dimensional parameters, thickness, width and or diameter, may preferably be up to about 500 μm, more preferably up to about 100 μm, such as about 50 μm.

In one embodiment, the sensor unit is a flexible sheet-formed unit with an average thickness of at least 5 times, preferably at least 50 times less than its average width, and/or the sensor unit is a flexible sheet-formed unit having an average thickness of at least 5 times, preferably at least 50 times less than its average length. As the sensor unit may have shapes with no unambiguous definition of width and length, e.g. rounded or circular shapes, it is generally preferred that such a sensor unit is in the form of a sheet-formed unit with an average thickness of at least 5 times, preferably at least 5 times less than its other dimensions including width, length and diameter. In case the sensor unit is of a rounded or circular shape, the following reference to width and length, respectively, means the shortest and the longest diameter, respectively, or stub diameter.

The term “flexible” used in relation to the sensor unit means that the sensor unit should be capable of deflecting, e.g. due to stress formed in the surface stress sensing element or due to amplification using an amplifier.

The connection line between the sensor unit and the pillar shaped primary substrate may be identified according to its material thickness i.e. the pillar shaped primary substrate is more rigid than the sensor unit, e.g. more than 3, 5 or 10 times as rigid as the sensor unit. The pillar shaped primary substrate may e.g. be thicker than the sensor unit, e.g. more than 3, 5 or 10 times or even more as thick as the sensor unit. The connection line between the sensor unit and the pillar shaped primary substrate is in the following denoted the stem of the sensor unit, and the tangent plane to the stem on the upper surface side of the sensor unit is measured on the sensor unit side of the stem. In situations where the sensor unit has a plane upper surface, the tangent plane is identical to the plane defined by the surface. In situations where the connection line between the sensor unit and the pillar shaped primary substrate is not straight, e.g. a rounded stem, there may be several tangent planes to the stem.

The tangent plane or planes to the stem of the upper surface of the sensor unit are in the following called the sensor unit plane or planes.

The sensor unit of the sensor according to the invention may be in the form of a sheet-formed unit with two major surfaces defined as the upper surface and the lower surface, respectively.

The upper surface of the sensor unit can be identified as the one of two major surfaces closest to the uppermost surface of the pillar shaped primary substrate.

The sensor unit is connected to the pillar shaped primary substrate so that it protrudes there from. In this embodiment, it is preferred that the upper surface or the sensor unit plane or planes have an angle to the uppermost surface of the pillar shaped primary substrate between 135° and 225°, preferably between 150° and 210°, such as between 165° and 195°. It is even more preferred that the upper surface of the sensor unit is substantially parallel to the uppermost surface of the pillar shaped primary substrate. In this connection it should be observed that “substantially parallel” includes a deviation of up to 10°, preferably up to 5°. Also, it should be observed that the uppermost surface of the pillar shaped primary substrate and the upper surface of the sensor unit preferably are in direct prolongation of each other. This embodiment is generally simpler to produce using standard photo-resist technique.

The lowermost surface of the pillar shaped primary substrate is defined as the surface opposite the uppermost surface, the lowermost surface is preferably substantially +−10° parallel with the uppermost surface. The pillar height is defined as the shortest distance between the uppermost surface and the lowermost surface measured perpendicular to the uppermost surface.

The pillar shaped primary substrate may have any cross sectional shape, e.g. round, oval, squared or other. In one embodiment, the cross sectional shape is essentially the same along the whole height of the pillar.

It is preferred that the wire or other parts of the electric communication line pass through the pillar shaped primary substrate material and exit the pillar shaped primary substrate at its lowermost surface to provide an electric communication line exit.

For optimising the design freedom the wire may preferably be integrated into said pillar shaped primary substrate so that the distance between the wire and the uppermost substrate surface differs along the wire, e.g. the distance between the wire and the uppermost pillar shaped primary substrate surface may e.g. differ along at least 50%, such as at least 75% or even more preferably all of the length of the integrated wire.

The uppermost surface of the pillar shaped primary substrate may preferably be substantially plane, where “substantially plane” should be interpreted as macroscopic plane surface, wherein the surface preferably is free of irregular cavities. It is particularly preferred that the wire passes through the pillar shaped primary substrate in a sum line of at least 45° or at least 65°, to the uppermost surface substrate. It should be observed that “sum line” means the straight line between the stem of the surface stress sensing element and the electric communication line exit.

It is even more preferred that the wire passes through the pillar shaped primary substrate in a sum line of about 90° to the uppermost surface of the pillar shaped primary substrate. By having an angle that is close to perpendicular to the uppermost surface of the pillar shaped primary substrate, the wire may pass through the pillar shaped primary substrate and exit at the lowermost surface of the pillar shaped primary substrate, which makes it possible to increase the number of sensor units of the sensor.

It is particularly preferred that both of the wires of said electric communication line pass through the pillar shaped primary substrate material in a straight line and exit the pillar shaped primary substrate through the lowermost surface, e.g. at an angle between the wire and the uppermost substrate surface which is about 90° +−10° in order thereby to provide electric communication line exits at the lowermost substrate surface. A wire having this angle to the uppermost substrate surface will in the following be denoted a vertical wire.

It is preferred that the wire in at least 50% of its pillar shaped primary substrate integrated length is perpendicular +−10°, preferably +−10°, to sensor unit plane or planes.

In one embodiment, both of the wires of the electric communication line pass through the pillar shaped primary substrate material in a substantially straight line, wherein “substantially straight” includes a partial deviation from the sum line of up to 10%, preferably up to 5% of the length of the sum line. The wires may preferably be vertical.

In one embodiment, the sensor unit may have any cantilever shape, e.g. as the cantilevers described in DK PA 2002 00125. The term “cantilever shape” is defined as a sheet formed unit linked to a substrate (or two substrates) along one or two opposite edge lines. The term “cantilever shape” thus also includes a bridge, as well as a traditional rectangular or leaf shaped cantilever.

In one embodiment, the sensor unit shaped as a cantilever with a longitudinal direction is linked in both of its longitudinal endings to form a cantilevered bridge.

In another embodiment, the cantilever is a traditional rectangular or leaf shaped cantilever linked to only one substrate. In the following this type of cantilever is referred to as cantilever with a free end.

It is particularly preferred that the sensor unit is a cantilever, preferably a cantilever in the form of a sheet-formed unit having a thickness which is thinner than its other dimensions.

In one embodiment, the cantilever is connected to the pillar shaped primary substrate and protrudes there from in one or more cantilever protruding directions.

The cantilever may preferably have a plane upper surface, and preferably also a plane lower surface when in a non-stressed state. It should, however, be observed that the cantilever may be curved or bended even in a non-stressed state.

In a preferred embodiment, the cantilever has a plane, non-curved upper surface in a non-stressed state. The upper surface of the cantilever is one of two major surfaces, where the upper surface is closest to the uppermost surface of the pillar shaped primary substrate measured at the stem of the cantilever.

The cantilever protrudes from the pillar shaped primary substrate in one or more cantilever protruding directions to provide a free edge of the cantilever. The two-dimensional cantilever shape defined as the shape surrounded by the cantilever free edge and the stem line along the connection to the pillar shaped primary substrate may be regular or irregular. The shape surrounded by the cantilever free edge and the stem line along the connection to the pillar shaped primary substrate may preferably be selected from the group consisting of square, rectangular, triangular, pentagonal, hexagonal, leaf shaped, circular and oval periphery.

In a particularly preferred embodiment of the sensor according to the invention where the sensor unit includes a cantilever, both of said wires in the pair of wires pass vertically through the pillar shaped primary substrate, such as in a sum angle which is substantially (+−10°) perpendicular to the substrate surface. The pillar shaped primary substrate is preferably shaped as a pillar, wherein the centre line of the pillar preferably is perpendicular +−20° to said uppermost surface of said pillar shaped primary substrate. The wires preferably pass through the pillar shaped primary substrate and exit the pillar at its lowermost surface.

The pillar may preferably be connected to a secondary substrate comprising a circuit for applying the voltage, said secondary substrate preferably being an electronic chip comprising contact pads corresponding with said wire exits.

Each pillar may comprise two or more cantilevers, wherein the wires of said cantilevers preferably pass vertically through the material of the pillar, and where the cantilevers protrude from the pillar. In this embodiment, it is preferred that the cantilevers have two-dimensional cantilever shapes which are substantially identical to each other, more preferably the two-dimensional cantilever shapes are preferably selected from the group consisting of square, rectangular, triangular, pentagonal, hexagonal and leaf shaped periphery.

Such pillar or pillars comprising one or more cantilevers will in the following also be denoted “free hanging cantilever element”.

In an alternative embodiment, the sensor unit is a bridge shaped cantilever, preferably in the form of a sheet-formed unit having a thickness which is thinner than its other dimensions, length and width, which bridge is connected to and stem from said pillar shaped primary substrate to link two primary substrates in a bridge.

The bridge may preferably have a plane upper surface and preferably also a plane lower surface when the bridge is in a non-stressed state. It should, however, be observed that the bridge may be curved or bended even in a non-stressed state.

In a preferred embodiment, the bridge has a plane, non-curved upper surface in a non-stressed state. The upper surface of the bridge is one of two major surfaces, where the upper surface is closest to the uppermost surface of the primary substrates wherein the wire(s) is/are integrated at the stem of the bridge into the primary substrates.

The bridge may preferably have a plane surface in a non-stressed state. In one embodiment, each primary substrate has an uppermost surface, which surface may preferably be substantially parallel (+−10°) with the upper surface of the bridge.

Both of said wires in the pair of wires may preferably pass through one of the primary substrates in a sum line which is substantially (+−10°) perpendicular to the primary substrates through which it passes. The centre line of the pillar shaped primary substrates may e.g. be perpendicular +−20° to said uppermost surface of the respective primary substrate. Thereby the wires pass through the substrate and exit the pillar shaped substrate(s) at its lowermost surface, and an improved design freedom is obtained.

The pillar shaped substrates may comprise two or more bridges, the wires of said bridges passing through the material of the pillars, and said bridges being connecting to two or more pillars.

Such pillar shaped primary substrate(s) in the shape of pillar or pillars comprising one or more bridges will in the following also be denoted “free hanging bridge element”.

Using the free hanging sensor unit element comprising pillar structures makes it possible to realise high-density two dimensional arrays. The sensor units in the array can be placed with the same spacing as used in DNA chips, e.g. as described in U.S. Pat. No. 6,254,827, and an array of sensor units can straightforwardly be used in the same type of applications as in the DNA chip. The signal from DNA chips is today read-out by the use of rather bulky optical detector systems and fluorescently labelled molecules. The present invention makes it possible to realise an array with the same performance but with a simple electrical and label free detection scheme. The sensor units may e.g. be functionalised with the same array sputter techniques as used in DNA chip production. Any other method may be used, e.g. as described in WO 0066266, WO 9938007, U.S. Pat. No. 5,156,810, WO 0036419 and WO 9631557, which publications are hereby incorporated by reference.

The free hanging sensor unit may also be placed in an interaction chamber, such as e.g. a flow channel. This can e.g. be used in micro liquid handling systems. The sensor therefore may comprise a fluid channel where the sensor units protrude into the fluid channel as disclosed in e.g. WO 9938007 and WO 0066266.

As also described in WO 0066266, the fluid channel may include an interaction chamber, and the sensor units e.g. in the form of free hanging sensor unit element may preferably be integrated into the wall of said interaction chamber.

According to the invention the space taken up by the wiring is reduced, and the number of sensor units e.g. cantilevers on the sensor (sensor chip) may be selected with higher freedom.

In one embodiment, an electrode can also be placed on the free hanging sensor element and electrically connected through the pillar. By controlling the potential of the electrodes, this can e.g. be used for immobilization of charged molecules or for direction of molecules since the electrode can form an electro endo-osmotic liquid flow towards the sensor.

The sensor according to the invention may in general include a secondary substrate supporting said primary substrate or substrates, e.g. in the form of free hanging sensor unit elements. The secondary substrate may comprise an electric communication line for applying a voltage over the respective pair of wire(s). The wires may e.g. be guided through the secondary substrate.

The secondary substrate may be an electronic chip comprising contact pads corresponding with said wire exits.

In a preferred embodiment, the sensor comprises a secondary substrate comprising an array of sensor units connected to the secondary substrate via primary substrates, preferably in the form of free hanging sensor unit element, wherein the wires are incorporated in the primary substrate material.

The secondary substrate may preferably be based on the same material as the pillar shaped primary substrate. In one embodiment, the pillar shaped primary substrate and the secondary substrate are of the same material.

The sensor may be in the form of a microchip, which means that none of its dimensions should exceed 10000 μm, preferably none of its dimensions should exceed 5000 μm.

The sensor according to the invention may preferably comprise at least one sensor unit having a target surface area, which area has been functionalised by linking of one or more functional groups comprising a detection ligand to said target surface area, said detection ligand being a member of a specific binding pair. Further information relating to this aspect can be found in WO 0066266 and DK patent application PA 200101724 which are hereby incorporated by reference. From these publications information relating to reference units can also be found.

It is thus preferred that the sensor comprises at least two sensor units, at least one of said sensor units being a reference units. The reference unit may preferably comprise a target surface area, which area has a surface chemistry which is different from the sensor unit for which the reference unit acts as reference, preferably said target surface area has been functionalised by linking of one or more functional groups, wherein said one or more functional groups linked to the surface area of said reference unit or its concentration are different from the sensor unit for which the reference unit acts as reference.

In one embodiment, one pillar is connected to both a sensor unit and its reference unit.

The sensor according to the invention may preferably be used for detection of substances in gasses or liquids, preferably in liquids wherein the substances include biomolecules such as RNA oligos, DNA oligos, PNA oligos, protein, peptides, hormones, blood components, antigen and antibodies.

The wire or wires may be integrated in the primary substrate using any technology, e.g. by casting or moulding the material of the primary substrate around the wire(s). The wire(s) may e.g. be layered between primary substrate material layers, which layers may be of similar or different materials. In a preferred embodiment, the primary substrate is prepared by e.g. using photo-resist technology as referred to above. The pillar shaped primary substrate may be prepared with a channel for the wire, or this channel may be provided afterwards. The channel should be applied through the pillar shaped primary substrate in a line as described above. Thereafter the channel is filled with a conducting material e.g. a metal e.g. by using electroplating.

The pillar shaped primary substrate may be prepared directly onto the secondary substrate, and the space (or part thereof) provided by the distance between the pillar shaped primary substrates may constitute the channel(s) or chamber(s) for liquid.

In one embodiment, the liquid channel is surrounding all of the pillar shaped substrates so that liquid applied therein can come into contact with the pillar walls and the sensor units.

In one embodiment, the sensor comprises a secondary substrate and a plurality of pillar shaped primary substrates, each of said pillar shaped primary substrates having an uppermost surface and a lowermost surface and a pillar wall surface. The pillar shaped primary substrates are connected to the secondary substrate at its lowermost surface. The sensor comprises a liquid chamber capable of containing a liquid so that liquid can be applied in said liquid chamber to surround one or more, preferably all of the pillar shaped primary substrates so that the pillar wall extending around said pillar shaped substrate and at least a part of the sensor unit connected to the pillar shaped substrates are contacted with the liquid.

In one embodiment, the sensor comprises a fluid channel, and the sensor units are partly or totally disposed in said fluid channel.

The sensor could be bonded to external electrical circuits using flip-chip technology, e.g. as described in U.S. Pat. No. 6,254,827 which is hereby incorporated by reference. The primary substrates in the form of pillars can be fabricated directly on an electronics chip.

### BRIEF DESCRIPTION OF DRAWINGS

FIG. 1 is a cross-sectional view of a prior art cantilever sensor.

FIGS. 2. are cross-sectional views of free hanging cantilever elements according to the invention shown with varying angles between cantilever and pillar.

FIG. 3 is a top view of a free hanging cantilever element according to the invention.

FIG. 4 is a cross-sectional view of a free hanging cantilever element placed on a secondary substrate.

FIGS. 5-7 are top views of different embodiments of free hanging cantilever elements according to the invention.

FIG. 8 is a cross-sectional view of a free hanging bridge element placed on a secondary substrate.

FIG. 9 is a top view of a free hanging cantilever element according to the invention wherein the cantilever comprises 4 individual cantilevers.

### DETAILED DESCRIPTION OF DRAWINGS

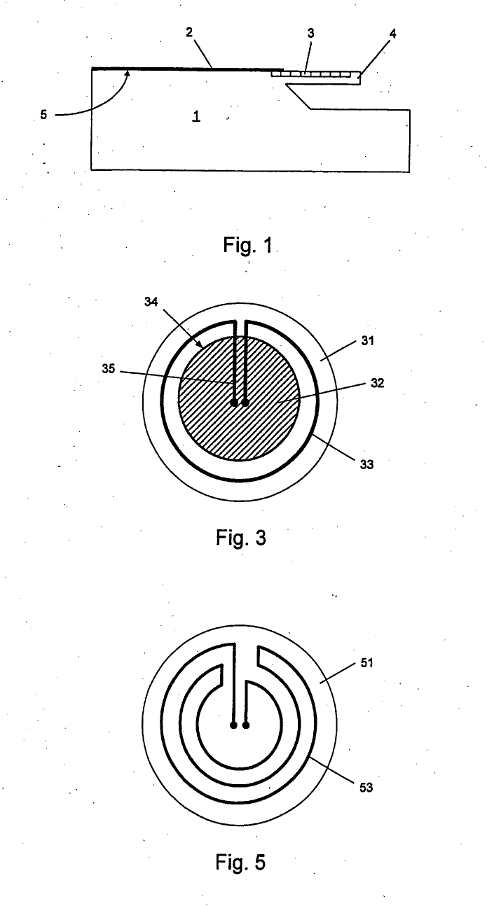

Prior art cantilever sensors for detecting substances, e.g. as disclosed in WO 9938007, generally have shapes as shown in FIG. 1. The shown prior art cantilever sensor comprises a primary substrate 1 and a sensor unit in the form of a cantilever 4 connected to the primary substrate 1. A surface stress sensing element 3, e.g. a piezoresistor such as a horseshoe-shaped piezoresistor, is placed on the surface of the cantilever 4, and a pair of parallel wires 2 is placed on the uppermost surface 5 of the primary substrate 1.

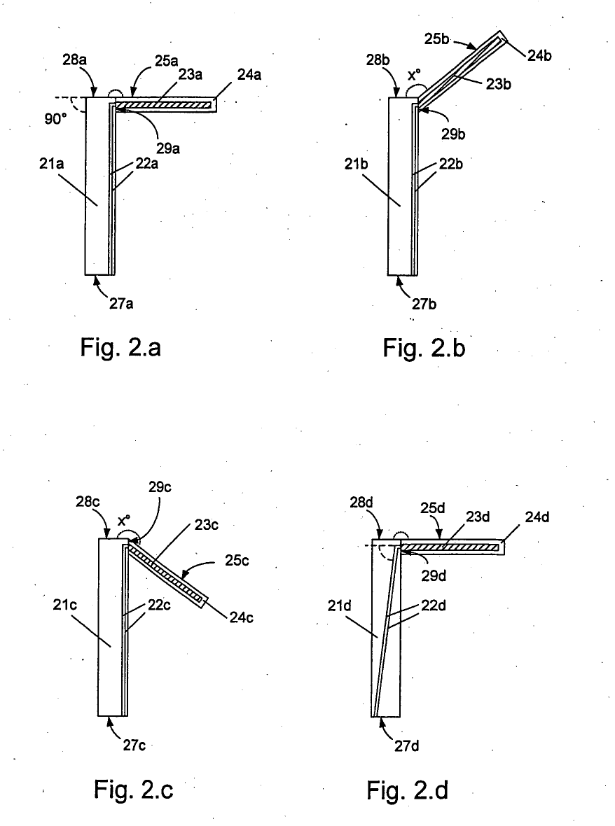

FIGS. 2a-2d show cross-sectional views of three different cantilever sensors according to the invention. The cantilever sensors comprise a primary substrate 21a, 21b, 21c, 21d and a cantilever 24a, 24b, 24c, 24d respectively. The respective cantilevers are connected to the respective pillars in a stem line 29a, 29b, 29c, In the cantilever sensor shown in FIG. 2a, a surface stress sensing element 23a, e.g. a piezoresistor such as a horseshoe-shaped piezoresistor, is incorporated into the cantilever unit 24a, and a pair of parallel wires 22a is integrated into the primary substrate as vertical wires, i.e. the wires 22a have an angle to the uppermost surface of the primary pillar shaped substrate 28a which is about 90° and they pass through the primary substrate and exit at its lowermost surface 27a. The uppermost surface of the primary substrate 28a and the upper surface of the cantilever unit 25a are parallel and in direct prolongation of each other.

In the cantilever sensor shown in FIG. 2b, a not shown surface stress sensing element, e.g. a piezoresistor such as a horseshoe-shaped piezoresistor, is incorporated into cantilever unit 24b as in FIG. 2a, and a pair of not shown parallel wires is integrated into the primary substrate as in FIG. 2a. The uppermost surface of the primary substrate 28b has an angle x° to the upper surface of the cantilever 25b which angle is less than 180, namely between 180 and 135°.

The cantilever sensor shown in FIG. 2c is identical to the cantilever sensor shown in FIG. 2b except that the uppermost surface of the primary substrate 28c has an angle x° to the upper surface of the cantilever 25c, which angle is higher than 180, namely between 180 and 225°.

In the cantilever sensor shown in FIG. 2d, a surface stress sensing element 23d, e.g. a piezoresistor such as a horseshoe-shaped piezoresistor, is incorporated into the cantilever unit 24d, and a pair of parallel wires 22d is integrated into the primary substrate at a small angle, e.g. 10° to the vertical direction, i.e. the wires 22a have an angle to the uppermost surface of the primary pillar shaped substrate 28d which is about 80° and they pass through the primary substrate and exit at its lowermost surface 27d. The uppermost surface of the primary substrate 28d and the upper surface of the cantilever unit 25d are parallel and in direct prolongation of each other.

FIG. 3 shows a top view of a cantilever connected to a pillar shaped primary substratum i.e. a free hanging cantilever element as defined herein. The cantilever unit 31 is ring-shaped and protrudes from the pillar 32 to which it is connected. The surface stress sensing element 33 in the form of a thin piezoresistor is arranged in a circle on the surface of the cantilever unit 31. It should be observed that it is generally preferred that the surface stress sensing element is embedded in the material of the sensor unit in order thereby to minimise undesired environmental interference such as short circuiting when brought into contact with liquid. The two ends of the surface stress sensing element are crossing the stem 34 of the cantilever unit and are passing into the pillar and the wires 35 pass further into the pillar and continue vertically through the pillars. As mentioned above, the piezoresistor 33 and the wires 35 may be of identical materials.

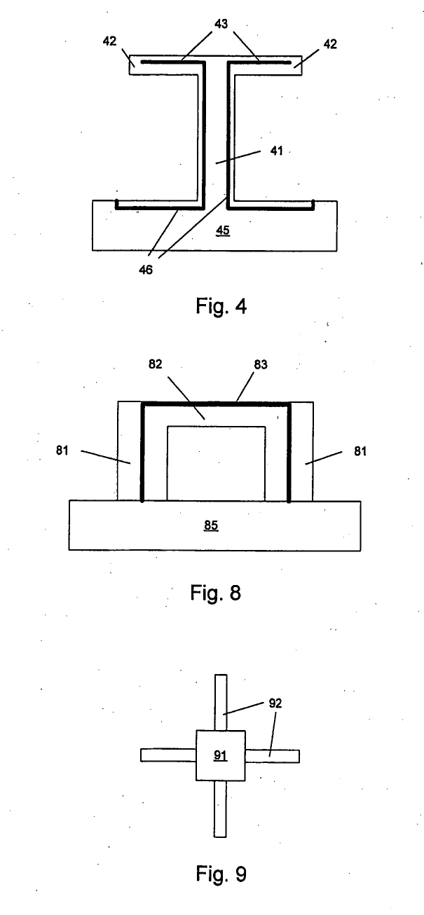

FIG. 4 is a cross-sectional view of a free hanging cantilever element disposed on a secondary substrate 45 e.g. a chip substrate. The free hanging cantilever element comprises a pillar 41 (primary substrate) and two individual cantilever units 42. Each of the cantilever units 42 comprises a piezoresistor 43 embedded in the cantilever sensor material. The piezoresistors are connected to wires 46, and the wires pass vertically through the pillar 41 and exit at the lower surface of the pillar 41 into the material of the secondary substrate 45.

The free hanging cantilever element shown in FIG. 5 is similar to the free hanging cantilever element shown in FIG. 3 except that the piezoresistor 53 is arranged in a different pattern on the cantilever unit 51. The stem of the cantilever 53 has not been marked in FIG. 5.

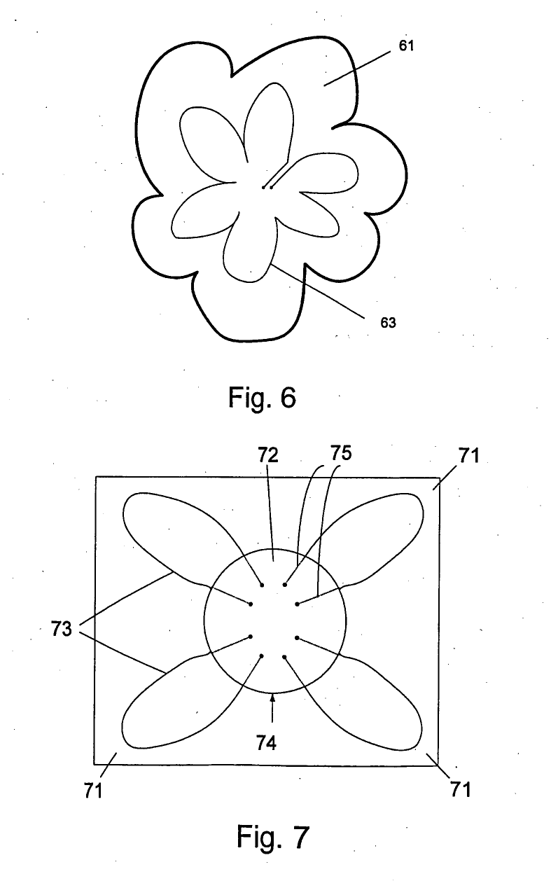

The free hanging flower-shaped cantilever element shown in FIG. 6 is similar to the free hanging cantilever element shown in FIGS. 3 and 5 except that the piezoresistor 63 is arranged in a different pattern on the cantilever unit 61. The stem of the cantilever 63 has not been marked in FIG. 6.

In a not shown variation of the flower-shaped sensor according to the invention shown in FIG. 6, each leaf of the flower is an individual cantilever unit comprising a piezoresistive element.

The free hanging cantilever element shown in FIG. 7 is another version similar to the free hanging cantilever element shown in FIGS. 3, 5 and 6. This free hanging cantilever element differs in that four cantilever units 71 are connected to the pillar 72 and protrude from the pillar 72. The cantilever units 71 are partly connected to each other and consequently the stress formation on one of the cantilever units may influence the measurement on another one of the sensor units. Each of the cantilever units 71 comprises a piezoresistor 73. The piezoresistor passes to the stem 74 of the respective cantilever unit 71, and the wires 75 pass vertically through the pillars.

FIG. 8 is a cross-sectional view of a free hanging bridge element placed on a secondary substrate, e.g. a chip substrate. The free hanging bridge element comprises two pillars 81 (primary substrates) and a bridge sensor unit 82 connected to the two pillars 81. The bridge unit comprises a piezoresistor 83 embedded in the bridge sensor material. The piezoresistor is connected to two wires, which may be of the same material as the piezoresistor, and the wires pass vertically through the pillars 81 and exit at the lower surface of the pillar 81 into the material of the secondary substrate 85.

FIG. 9 is a top view of a free hanging cantilever element with 4 individual cantilever units 92 connected to the pillar 91. The cantilever units comprise a not shown surface stress sensing element incorporated into the material of the cantilever units 92, and the pillar 91 comprises not shown vertical wires.

### EXAMPLE

An example of the fabrication of a high-density two-dimensional array of cantilever-based sensors placed on stand-alone pillars is described briefly below.

The circuits for connecting individual pillars are defined on a secondary substrate in the form of a separate chip using standard microelectronics techniques. Next, the electronics chip is coated with an insulating film, e.g. the polymer SU8. Windows are opened in the SU8 above the electrical contact pads on the electronics chip. A second layer of SU8 is spin coated on the chip and pillars centred on the contact pads are defined. One or more vertical channels are running inside the pillars, thereby creating contact to the electronics chip below. These channels are then filled with a metal, e.g. by electroplating. After that, a photoresist is spun on the structures and planarized so that the resist has about the same height as the pillars. Windows are opened in the resist to the electroplated wires. The resist is hard baked. The bottom of the cantilever is defined in SU8 and the piezoresistors connected to the electroplated vertical wires are defined in gold. Finally, the top part of the cantilever is defined in SU8, hereby completely encapsulating the resistors. The planarized resist is dissolved resulting in free hanging cantilever structures.

1. A sensor for detecting a substance in a liquid, said sensor comprising a primary substrate and a sensor unit connected to said primary substrate, said primary substrate being shaped as a pillar, said sensor comprising detecting means for detecting a change of stress or mass generated on a surface area of the sensor unit, and an electric communication line for applying a voltage over said detection means, at least one of said wires being integrated in said pillar shaped primary substrate.

2. A sensor according to claim 1 wherein said sensor unit is a flexible unit in the form of a cantilever, such as a cantilever connected to one pillar shaped substrate, and a cantilever connected to two pillar shaped substrates e.g. a bridge.

3. A sensor according to claim 1 wherein said sensor unit is a flexible sheet-formed unit having an average thickness which is less than both its average length and its average width.

4. A sensor according to claim 1 wherein said means for detecting a change of stress generated on a surface area of the sensor unit is in the form of a surface stress sensing element integrated in the sensor unit, said electric communication line including a pair of wires for applying an electrical field over the surface stress sensing element, said surface stress sensing element preferably being selected from the group consisting of a piezoelectric element, a strain gauge, a Si or C nanotube, a capacitor and a piezoresistor.

5. A sensor according to claim 1 wherein said means for detecting a change of stress generated on a surface area of the sensor unit is in the form of a laser system.

6. A sensor according to claim 1 wherein said pillar shaped primary substrate has an uppermost surface and a lowermost surface and a height defined as the shortest distance between said uppermost and lowermost surfaces, which sensor unit is a flexible sheet-formed unit having two major surfaces, said sensor unit being connected to said primary substrate so that it protrudes from the primary substrate, said upper surface of said sensor unit having an angle to the uppermost surface of said primary substrate between 135° and 225°, said upper surface of said sensor unit preferably being substantially parallel to the uppermost surface of said primary substrate, said uppermost surface of said primary substrate and said upper surface of the sensor unit preferably being in direct prolongation of each other.

7. A sensor according to claim 6 wherein said uppermost surface of the primary substrate is substantially plane and said electric communication line passes through the primary substrate in a sum line having an angle of at least 45° or at least 65°, such as about 90° to the uppermost surface of the primary substrate.

8. A sensor according to claim 1 wherein one or both of the wires of said electric communication line pass through the primary substrate and exit the primary substrate to provide electric communication line exit(s) at the lowermost surface of the primary substrate, said lowermost surface of the primary substrate being connected to a secondary substrate.

9. A sensor according to claim 1 wherein one or both of the wires of said electric communication line pass through the primary substrate material in a substantially straight line.

10. A sensor according to claim 4 wherein said surface stress sensing element in the form of a piezoresistor or a strain gauge comprises or preferably consists of a material selected from the group consisting of amorph silicon, polysilicon, single crystal silicon, metal or metal containing composition, e.g. gold, AlN, Ag, Cu, Pt and Al conducting polymers, such as, doped octafunctional epoxidized novalac e.g. doped SU-8, and composite materials with an electrically non-conducting matrix and a conducting filler, wherein the filler preferably is selected from the group consisting of amorph silicon, polysilicon, single crystal silicon, metal or metal containing composition, e.g. gold, AlN, Ag, Cu, Pt and Al, semi-conductors, carbon black, carbon fibres, particulate carbon, carbon nanowires, silicon nanowires.

11. A sensor according to claim 4 wherein said capacitor in the form of two conducting elements of e.g. metal or conductive polymers is separated in a distance of up to about 5 □m from each other by a dielectricum selected from the group consisting of liquid, gas or solids e.g. air, and octafunctional epoxidized novalac e.g. SU-8.

12. A sensor according to claim 1 wherein said primary substrate comprises one or more of the materials selected from the group consisting of silicon, silicon nitride, silicon oxide, metal, metal oxide, glass and polymer, wherein the group of polymers preferably includes epoxy resin e.g. an octafunctional epoxidized novalac, polystyrene, polyethylene, polyvinylacetate, polyvinylcloride, polyvinylpyrrolidone, polyacrylonitrile, polymethylmetacrylate, polytetrafluoroethylene, polycarbonate, poly-4-methylpentylene, polyester, polypropylene, cellulose, nitrocellulose, starch, polysaccarides, natural rubber, butyl rubber, styrene butadiene rubber and silicon rubber.

13. A sensor according to claim 1 wherein said sensor unit is based on a material included in the primary substrate, preferably said sensor unit is based on the same material as that of the primary substrate, more preferably said sensor unit being integrated with said primary substrate.

14. A sensor according to claim 1 wherein said primary substrate and said secondary substrate are of the same material.

15. A sensor according to claim 1 wherein said cantilever is connected to one pillar shaped primary substrate and protrudes from the primary substrate in one or more cantilever protruding directions to provide a free edge of said cantilever, said cantilever having a two-dimensional cantilever shape defined as the shape surrounded by the cantilever free edge and the stem line along the connection to the pillar shaped primary substrate, which shape may be regular or irregular, and preferably is selected from the group consisting of square, rectangular, triangular, pentagonal, hexagonal, leaf shaped, circular and oval periphery.

16. A sensor according to claim 1 wherein said sensor unit in the form of a cantilever is connected to two pillars shaped primary substrates to thereby form a bridge.

17. A sensor according to claim 15 wherein said primary substrate has an uppermost surface or said primary substrates have uppermost surfaces, said uppermost substrate surface(s) being substantially parallel with the upper surface of the cantilever when the cantilever is in a non stressed state.

18. A sensor according to claim 15 wherein both of said wires in the pair of wires pass through the primary substrate or substrates in a sum line having an angle which is substantially perpendicular to the uppermost substrate surface(s), the centre line of the pillar shaped primary substrate(s) preferably being perpendicular +−20° to uppermost surface thereof, which wires pass through the primary substrate and exit the pillar shaped primary substrate(s) at its lowermost surface.

19. A sensor according to claim 18 wherein said pillar shaped primary substrate(s) is/are connected to a secondary substrate comprising a circuit for applying the voltage, said secondary substrate preferably being an electronic chip comprising contact pads corresponding with said wire exits.

20. A sensor according to claim 17 wherein said pillar shaped primary substrate(s) is/are connected to two or more cantilevers, the wires of which cantilevers pass through the pillar shaped primary substrate(s), said cantilevers preferably having a two-dimensional cantilever shape which is substantially identical to each other, more preferably said two-dimensional cantilever shape preferably being selected from the group consisting of square, rectangular, triangular, pentagonal, hexagonal and leaf shaped periphery.

21. A sensor according to claim 1 further comprising a secondary substrate supporting said pillar shaped primary substrate or substrates, said secondary substrate comprising an electric supply line for supplying an electric field over the respective pair(s) of wires, said wires preferably being guided through the secondary substrate.

22. A sensor according to claim 21 wherein said secondary substrate is an electronic chip comprising contact pads corresponding with said wire exits.

23. A sensor according to claim 21 wherein said secondary substrate carries an array of pillar shaped primary substrates carrying sensor units connected thereto, wherein the wires are incorporated in the primary substrates.

24. A sensor according to claim 1 wherein said sensor comprises a secondary substrate and a plurality of pillar shaped primary substrates, each of said pillar shaped primary substrates having an uppermost surface and a lowermost surface, and a pillar wall surface, said pillar shaped primary substrates being connected to said secondary substrate at its lowermost surface, said sensor comprising a liquid chamber capable of containing a liquid so that liquid can be applied in said liquid chamber to surround one or more, preferably all of said pillar shaped primary substrates so that the pillar wall extending around said pillar shaped substrate and at least a part of the sensor unit connected to the pillar shaped substrates are contacted with the liquid.

25. A sensor according to claim 1 wherein said sensor further comprises a fluid channel, said sensor units partly or totally being disposed in said fluid channel, said pillar shaped primary substrates preferably being disposed in said fluid channel.

26. A sensor according to claim 1 wherein said sensor comprises at least one sensor unit having a target surface area, which area has been functionalised by linking of one or more functional groups comprising a detection ligand to said target surface area, said detection ligand being a member of a specific binding pair.

27. A sensor according to claim 1 wherein the sensor comprises at least two sensor units, at least one of said sensor units being a reference unit.

28. A sensor according to claim 27 wherein said reference unit comprises a target surface area, which area has a surface chemistry different from the sensor unit for which the reference unit acts as reference, preferably said target surface area has been functionalised by linking of one or more functional groups, wherein said one or more functional groups linked to the surface area of said reference unit or its concentration are different from the sensor unit for which the reference unit acts as reference.
